(19) United States
(12) Patent Application Publication
(10) Pub. No.: US 2014/0180044 A1
(43) Pub. Date: Jun. 26, 2014

(54) METHODS AND SYSTEMS FOR DETERMINING SIGNAL QUALITY OF A PHYSIOLOGICAL SIGNAL

(71) Applicant: COVIDIEN LP, Mansfield, MA (US)

(72) Inventors: Paul Stanley Addison, Edinburgh (GB); James Nicholas Watson, Dunfermline (GB)

(73) Assignee: Covidien LP, Mansfield, MA (US)

(21) Appl. No.: 13/726,072

(22) Filed: Dec. 22, 2012

(51) Int. Cl. A61B 5/1455

(52) U.S. Cl. CPC A61B 5/14551
USPC 600/324

(57) ABSTRACT

A physiological monitoring system may use photonic signals at one or more wavelengths to determine physiological parameters. The system may receive a photoplethysmograph signal, and generated a difference signal based on the photoplethysmograph signal. The system may specify a segment of the photoplethysmograph signal and a segment of the difference signal. The system may associate each value of the segment of the photoplethysmograph signal to a corresponding value of the segment of the difference signal to generate associated value pairs. The system may compare the associated value pairs to a reference characteristic, and determine a signal quality metric based on the comparison.

The present disclosure relates to determining signal quality, and more particularly relates to determining signal quality using an attractor.

### SUMMARY

Methods and systems are provided for determining signal quality of a physiological signal.

In some embodiments, a method for determining signal quality of a physiological signal may include receiving a photoplethysmograph signal. The method may further include generating a difference signal based on the photoplethysmograph signal. The method may further include specifying a segment of the photoplethysmograph signal including a first plurality of values, and specifying a segment of the difference signal including a second plurality of values. The method may further include associating each value of the first plurality of values with a corresponding value of the second plurality of values to generate a plurality of associated value pairs, and comparing the plurality of associated value pairs to a reference characteristic. The method may further include determining a signal quality metric based on the comparison.

In some embodiments, a system for determining signal quality of a physiological signal may include processing equipment. The processing equipment may be configured to receive a photoplethysmograph signal, and generate a difference signal based on the photoplethysmograph signal. The processing equipment may be further configured to specify a segment of the photoplethysmograph signal including a first plurality of values, and specify a segment of the difference signal including a second plurality of values. The processing equipment may be further configured to associate each value of the first plurality of values with a corresponding value of the second plurality of values to generate a plurality of associated value pairs, and compare the plurality of associated value pairs to a reference characteristic. The processing equipment may be further configured to determine a signal quality metric based on the comparison.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system, in accordance with some embodiments of the present disclosure;

FIG. 2 is a block diagram of the illustrative patient monitoring system of FIG. 1 coupled to a patient, in accordance with some embodiments of the present disclosure;

FIG. 3 shows a block diagram of an illustrative signal processing system in accordance with some embodiments of the present disclosure;

FIG. 4 shows an illustrative periodic signal, an illustrative difference signal, and an attractor generated thereof, in accordance with some embodiments of the present disclosure;

FIG. 5 shows a plot of an illustrative PPG signal, in accordance with some embodiments of the present disclosure;

FIG. 6 shows a plot of an illustrative difference signal derived from the PPG signal of FIG. 5, in accordance with some embodiments of the present disclosure;

FIG. 7 shows a plot of an illustrative attractor generated based on the PPG signal of FIG. 5 and the difference signal of FIG. 6, in accordance with some embodiments of the present disclosure;

FIG. 8 is a flow diagram of illustrative steps for determining a signal quality metric, in accordance with some embodiments of the present disclosure;

FIG. 9 shows a plot of the illustrative attractor of FIG. 7 and a reference characteristic, in accordance with some embodiments of the present disclosure;

FIG. 10 is a histogram 1000 of intersection locations of the attractor and reference curve of FIG. 9, in accordance with some embodiments of the present disclosure;

FIG. 11 shows a plot of an illustrative attractor in three-dimensions generated using associated value triples, and a reference characteristic, in accordance with some embodiments of the present disclosure; and

FIG. 12 shows intersections of the attractor and the reference characteristic of FIG. 11, viewed normal to the reference characteristic, in accordance with some embodiments of the present disclosure.

### DETAILED DESCRIPTION OF THE FIGURES

The present disclosure is directed towards determining a signal quality metric based on an attractor generated from segments of a photoplethysmograph signal. The PPG signal may be generated using, for example, an oximeter such as a pulse oximeter.

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and blood pressure.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the blood pressure monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a pulse rate and when each individual pulse occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=Iₒ(λ)exp(−(sβₒ(λ)+(1−s)βᵣ(λ))l(t)) (1)

where:
λ=wavelength;
t=time;
I=intensity of light detected;
I₀=intensity of light transmitted;
s=oxygen saturation;
β₀, βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log Iₒ−(sβₒ+(1−s)βᵣ)l. (2)

2. Eq. 2 is then differentiated with respect to time to yield

$$\frac{d log I}{d t} = - ( s {β}_{o} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{o} ( {λ}_{IR} ) - {β}_{r} ( {λ}_{IR} ) ) - \\ \frac{d log I ( {λ}_{IR} )}{d t} ( {β}_{o} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}). (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

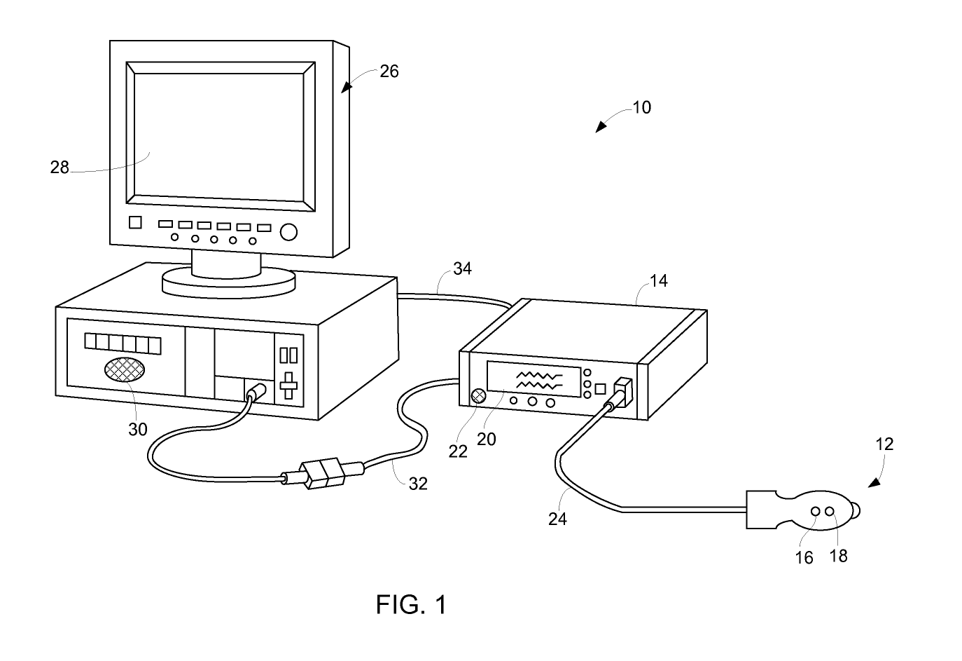

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According to another embodiment, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood pressure, blood oxygen saturation) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the monitor 14 includes a blood pressure monitor. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may be allow communication between monitor 14 and sensor unit 12.

In the illustrated embodiment, system 10 includes a multi-parameter patient monitor 26. The monitor 26 may include a cathode ray tube display, a flat panel display (as shown) such as a liquid crystal display (LCD) or a plasma display, or may include any other type of monitor now known or later developed. Multi-parameter patient monitor 26 may be configured to calculate physiological parameters and to provide a display 28 for information from monitor 14 and from other medical monitoring devices or systems (not shown). For example, multi-parameter patient monitor 26 may be configured to display an estimate of a patient's blood oxygen saturation generated by monitor 14 (referred to as a “SpO₂” measurement), pulse rate information from monitor 14 and blood pressure from monitor 14 on display 28. Multi-parameter patient monitor 26 may include a speaker 30.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by a battery (not shown) or by a conventional power source such as a wall outlet.

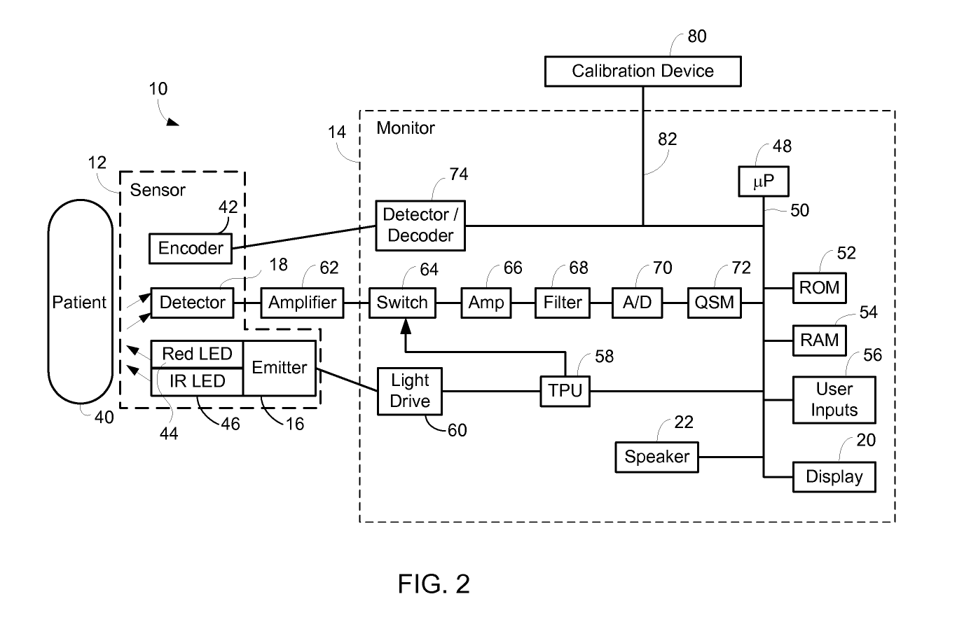

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor emits only a Red light while a second emits only an IR light. In a further example, the wavelengths of light used are selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which, for example, blood pressure and other measurements may be determined based at least in part on the signal or signals received at sensor unit 12. For example, some pulse oximetry sensors rely on equations to relate an area under a portion of a photoplethysmograph (PPG) signal corresponding to a physiological pulse to determine blood pressure. These equations may contain coefficients that depend upon a patient's physiological characteristics as stored in encoder 42. Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics. In some embodiments, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as SpO₂, pulse rate, and/or blood pressure, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

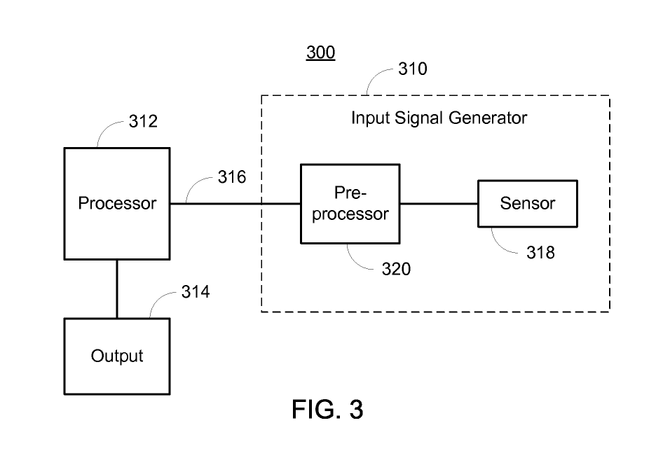

FIG. 3 is an illustrative signal processing system 300 in accordance with an embodiment that may implement the signal processing techniques described herein. In some embodiments, signal processing system 300 may be included in a patient monitoring system (e.g., patient monitoring system 10 of FIGS. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be an oximeter and input signal 316 may be a PPG signal. In an embodiment, pre-processor 320 may be any suitable signal processing device and input signal 316 may include one or more PPG signals and one or more other physiological signals, such as an electrocardiogram (ECG) signal. It will be understood that input signal generator 310 may include any suitable signal source, signal generating data, signal generating equipment, or any combination thereof to produce signal 316. Signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, signal 316 may include PPG signals corresponding to one or more light frequencies, such as a Red PPG signal and an IR PPG signal. In some embodiments, signal 316 may include signals measured at one or more sites on a patient's body, for example, a patient's finger, toe, ear, arm, or any other body site. In some embodiments, signal 316 may include multiple types of signals (e.g., one or more of an ECG signal, an EEG signal, an acoustic signal, an optical signal, a signal representing a blood pressure, and a signal representing a heart rate). Signal 316 may be any suitable biosignal or signals, such as, for example, electrocardiogram, electroencephalogram, electrogastrogram, electromyogram, heart rate signals, pathological sounds, ultrasound, or any other suitable biosignal. The systems and techniques described herein are also applicable to any dynamic signals, non-destructive testing signals, condition monitoring signals, fluid signals, geophysical signals, astronomical signals, electrical signals, financial signals including financial indices, sound and speech signals, chemical signals, meteorological signals including climate signals, any other suitable signal, and/or any combination thereof.

In some embodiments, signal 316 may be coupled to processor 312. Processor 312 may be any suitable software, firmware, hardware, or combination thereof for processing signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may locate one or more reference points on one or more signals, which may be found by performing mathematical calculations on the signal. In a further example, processor 312 may locate one or more fiducial points on one or more signals, and compute one or more of a pulse rate, respiration rate, blood pressure, or any other suitable physiological parameter. Processor 312 may perform any suitable signal processing of signal 316 to filter signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the patient. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 312 to, for example, store fiducial information corresponding to physiological monitoring. In some embodiments, processor 312 may store physiological measurements or previously received data from signal 316 in a memory device for later retrieval. In some embodiments, processor 312 may store calculated values, such as a blood pressure, a blood oxygen saturation, a pulse rate, a fiducial point location or characteristic, or any other calculated values, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIGS. 1 and 2) in which, for example, input signal generator 310 may be implemented as part of sensor unit 12 (FIGS. 1 and 2) and monitor 14 (FIGS. 1 and 2) and processor 312 may be implemented as part of monitor 14 (FIGS. 1 and 2). In some embodiments, portions of system 300 may be configured to be portable. For example, all or part of system 300 may be embedded in a small, compact object carried with or attached to the patient (e.g., a watch, other piece of jewelry, or a smart phone). In some embodiments, a wireless transceiver (not shown) may also be included in system 300 to enable wireless communication with other components of system 10 (FIGS. 1 and 2). As such, system 10 (FIGS. 1 and 2) may be part of a fully portable and continuous patient monitoring solution. In some embodiments, a wireless transceiver (not shown) may also be included in system 300 to enable wireless communication with other components of system 10. For example, pre-processor 320 may output signal 316 over BLUETOOTH, 802.11, WiFi, WiMax, cable, satellite, Infrared, or any other suitable transmission scheme. In some embodiments, a wireless transmission scheme may be used between any communicating components of system 300.

Pre-processor 320 or processor 312 may determine the locations of pulses within a periodic signal 316 (e.g., a PPG signal) using a pulse detection technique. For ease of illustration, the following pulse detection techniques will be described as performed by processor 312, but any suitable processing device (e.g., pre-processor 320) may be used to implement any of the techniques described herein.

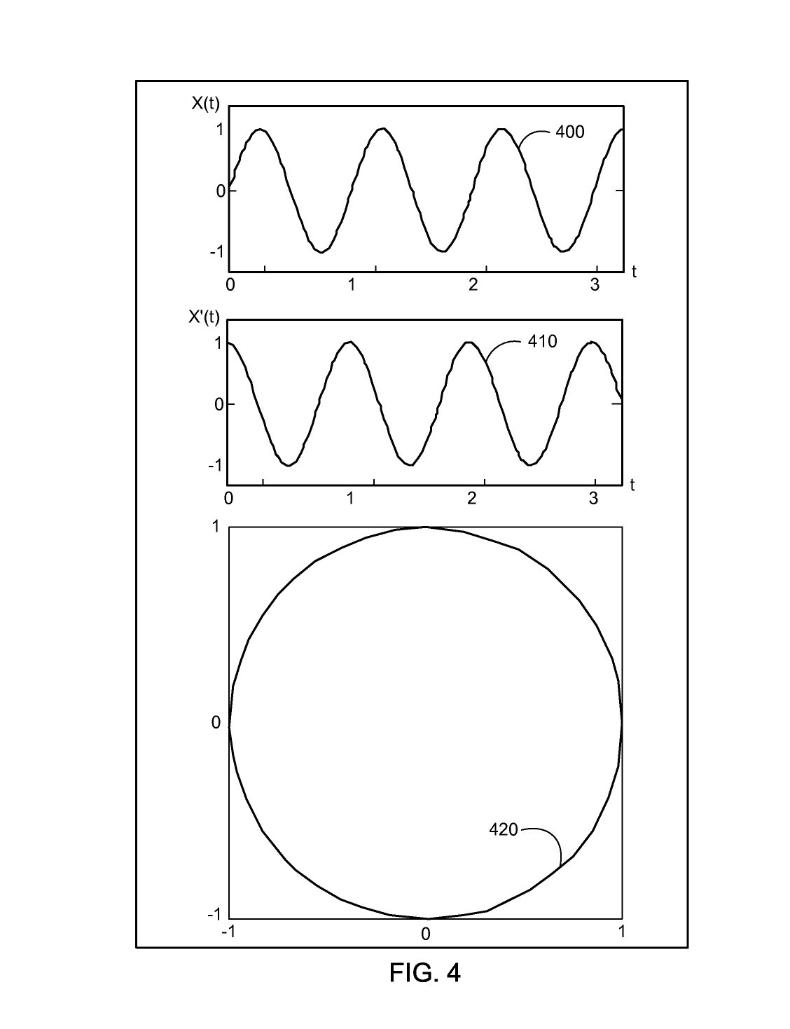

FIG. 4 shows an illustrative periodic signal 400, an illustrative difference signal 410, and an attractor 420 generated thereof, in accordance with some embodiments of the present disclosure. Periodic signal 400, a sine wave having a period and amplitude of one, is a relatively simple periodic signal for purposes of illustration. Difference signal 410, is a cosine wave having a period and amplitude of one, derived by taking the derivative of signal 400. In some embodiments, a set of value pairs may be generated by pairing values of periodic signal 400, for example at discrete time t values, with values of difference signal 410 at the same discrete time t values. The set of value pairs is referred to herein as an attractor. Attractor 420 was generated by pairing corresponding values of signal 400 and difference signal 410. In some embodiments, an attractor may be interrogated, using processing equipment, to determine the stability of the attractor, which may be indicative of the variability in the PPG signal from cycle to cycle. Details regarding attractors, and analysis thereof, may be found in the book “Fractals and Chaos: An illustrated Course” by Paul S. Addison, 1997, which is hereby incorporated by reference herein in its entirety.

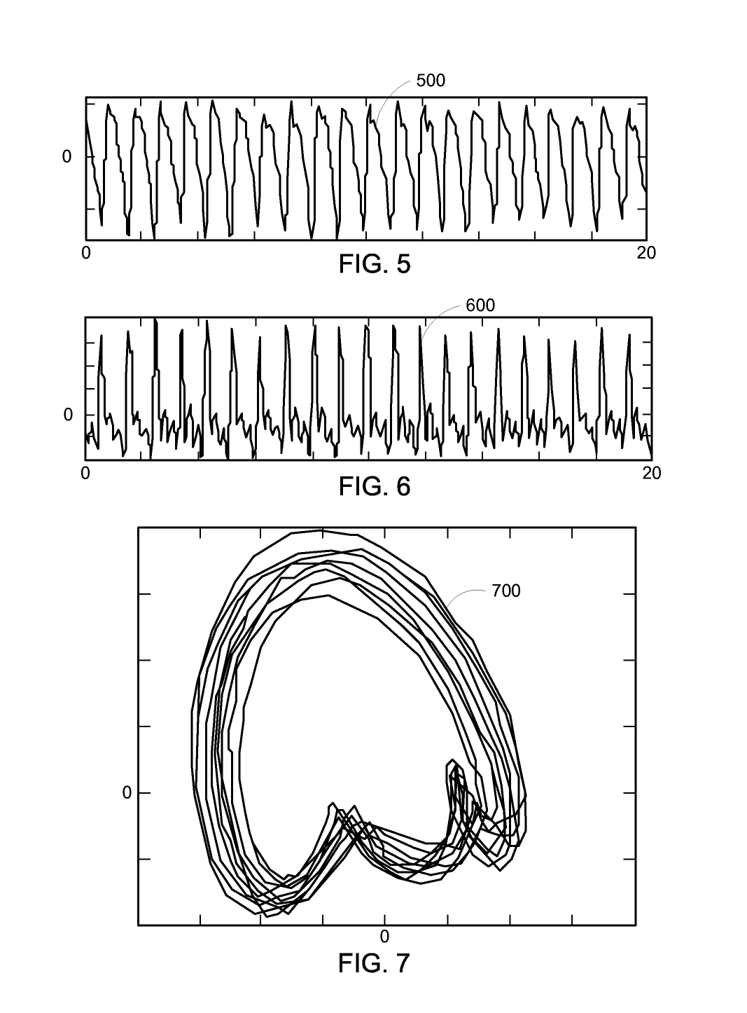

FIG. 5 shows a plot of an illustrative PPG signal 500, in accordance with some embodiments of the present disclosure. PPG signal 500 includes multiple pulse waves, centered about zero. FIG. 6 shows a plot of an illustrative difference signal 600 derived from PPG signal 500 of FIG. 5, in accordance with some embodiments of the present disclosure. In some embodiments, for example, a difference signal such as difference signal 600 may be generated from a PPG signal using a forward difference, a central difference, a backward difference, a derivative, or any other suitable technique. FIG. 7 shows a plot of an illustrative attractor 700 generated based on PPG signal 500 of FIG. 5 and difference signal 600 of FIG. 6, in accordance with some embodiments of the present disclosure. The attractor is generated by pairing corresponding values of PPG signal 500 and difference signal 600. Attractor 700 includes a collection of associated value pairs (x(t), y(t)), where:

x(t)=f(t) (14)

y(t)=f′(t) (15)

in which f(t) is the value of PPG signal 500 at time t, and f′(t) is the value of difference signal 600 at time t. Attractor 700 is not a circle, due to the shape of pulse waves of PPG signal 700. Also, attractor 700 is not a closed curve, but rather includes a sequence of nearly closed shapes (each corresponding to one period, referred to as a cycle), which are substantially similar but not identical, having some variation due to variability in PPG signal 500. The variability in the sequence of shapes provides an indication of the variability of the signal. In some embodiments, an attractor may be analyzed to determine the stability (e.g., similarity, or lack of variation, from cycle to cycle) of the attractor. It will be understood that processing equipment need not plot an attractor to analyze the attractor, and FIGS. 5-7 and 9-12 are provided as graphical examples.

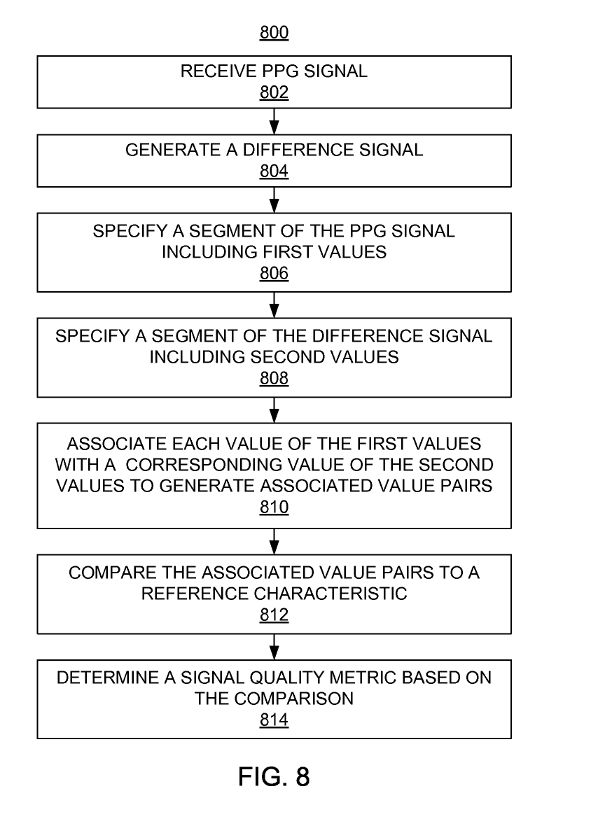

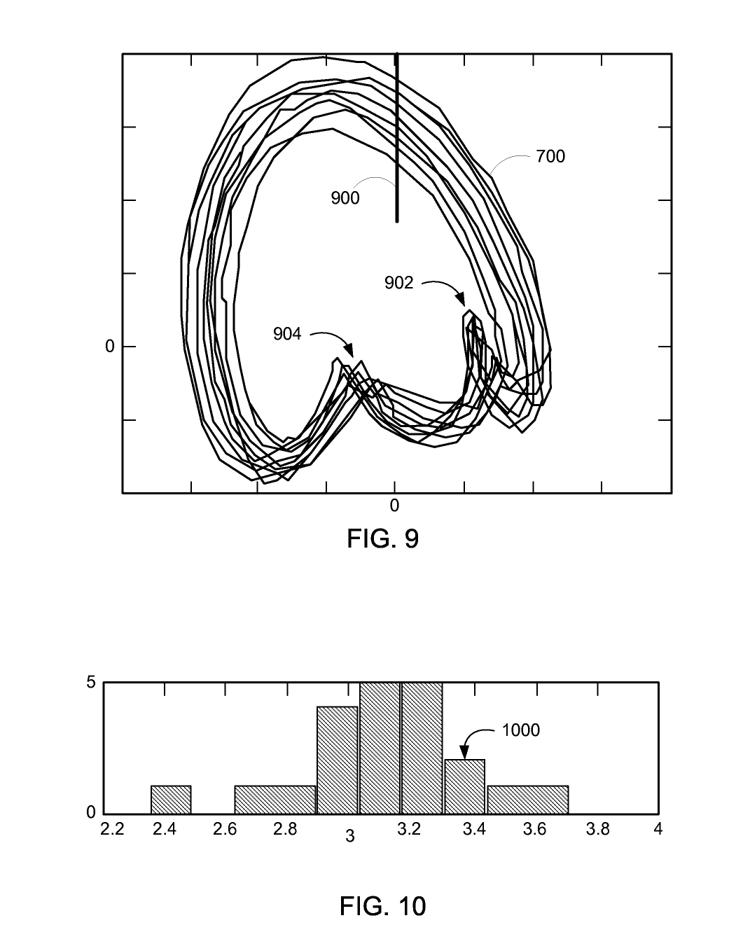

FIG. 8 is a flow diagram 800 of illustrative steps for determining signal quality based on a subset of associated value pairs, in accordance with some embodiments of the present disclosure. FIGS. 9-10, which provide graphical examples of some techniques of the present disclosure, will be referenced in the context of flow diagram 800.

Step 802 may include processing equipment receiving a PPG signal from a physiological sensor, memory, any other suitable source, or any combination thereof. For example, referring to system 300 of FIG. 3, the processing equipment may receive a window of physiological data from input signal generator 310. Sensor 318 of input signal generator 310 may be coupled to a subject, and may detect physiological activity such as, for example, RED and/or IR light attenuation by tissue, using a photodetector. In some embodiments, physiological signals generated by input signal generator 310 may be stored in memory (e.g., RAM 54 of FIG. 2, QSM 72 and/or other suitable memory) after being pre-processed by pre-processor 320. In such cases, step 802 may include recalling data from the memory for further processing. In some embodiments, the processing equipment may filter the PPG signal. For example, the processing equipment may apply a high-pass filter (e.g., having a cutoff frequency below the expected heart rate) to reduce or substantially remove baseline changes and other low-frequency artifacts. In a further example, the processing equipment may apply a low-pass filter (e.g., having a cutoff frequency above the expected heart rate) to reduce or substantially remove higher frequency noise or features. In a further example, the processing equipment may apply a bandpass filter to reduce or substantially remove low and high frequency artifacts and features. In some embodiments, the illustrative steps of flow diagram 800 may be based on a Red PPG signal, IR PPG signal, a derivative thereof, a processed signal derived thereof, or any combination thereof.

Step 804 may include processing equipment generating a difference signal based on the received PPG signal of step 802 (e.g., by calculating a sequence of difference values between adjacent values of the PPG signal). In some embodiments, the processing equipment may perform a subtraction between values of adjacent values. In some embodiments, the processing equipment may calculate the differences by calculating a first derivative of the PPG signal. For example, the processing equipment may compute forward differences, backward differences, or central differences between each pair of adjacent values to generate a difference signal. In a further example, the processing equipment may compute a numerical derivative at each value of the PPG signal, generating a difference signal. Any suitable difference technique may be used by the processing equipment to generate the difference signal. In some embodiments, the processing equipment may normalize, shift, or otherwise scale the difference signal. For example, the processing equipment may normalize both the PPG signal and difference signal to vary between −1 and 1. In a further example, the PPG signal, difference signal, or both, may be normalized based on the respective standard deviation and/or mean energy of the respective signal.

Step 806 may include the processing equipment specifying a segment of the PPG signal of step 802. In some embodiments, the segment of the PPG signal may include multiple values (e.g., a particular number of values). In some embodiments, selecting the segment of the PPG signal may include specifying indices of values of the PPG signal. The length of the segment, in time or sample number, may be any suitable length. For example, the length may be equal to a period corresponding to a physiological rate (e.g., a heart rate), or a multiple thereof. The segment of the PPG signal will be referred to herein as a first segment.

Step 808 may include the processing equipment specifying a segment of the difference signal of step 804. The segment of the difference signal includes the same number of samples points as the segment of the PPG signal specified at step 806. In some embodiments, the segments of the PPG signal and the difference signal are coincident in time. For example, a segment of the PPG signal may correspond to a time interval T₀-T_{N}, and the segment of the difference signal may also correspond to the time interval T₀-T_{N}, where T₀ is a starting time and T_{N} is an ending time. Note that when the first and second segments are coincident in time, transient noise artifacts may be localized in the associated value pairs (e.g., localized to a particular region of the attractor). In some embodiments, selecting the segment of the difference signal may include specifying indices of values in the difference signal. The segment of the difference signal will be referred to herein as a second segment.

Step 810 may include the processing equipment associating each value of the first segment with a corresponding value of the second segment to generate associated value pairs. The associated value pairs include a first value and a second value from the PPG signal and difference signal. As referred to herein, the first value will correspond with the first segment and the second value will correspond to the second segment, although in some embodiments the order may be switched. In some embodiments, the associated value pairs are generated by associating values of the first and second segments by relative indices within the respective segments. For example, the first point of the first segment may be associated with the first point of the second segment, the second point of the first segment may be associated with the second point of the second segment, and so on. In some embodiments, the processing equipment may store the associated value pairs, indices corresponding to the associated value pairs, or both.

Step 812 may include the processing equipment comparing the associated value pairs, or a subset thereof, to a reference characteristic. In some embodiments, the reference characteristic may correspond to a line, polynomial of degree 2 or higher, a piecewise curve, any other suitable curve at any suitable orientation relative to the attractor, or any combination thereof, when considered in two-dimensional space. For example, when considered in two-dimensional space, the reference characteristic may be a horizontal line, a vertical line, an oblique line, or piecewise combination thereof. In some embodiments, the comparison may include identifying intersections of the associated value pairs and the reference characteristic. For example, the associated value pairs nearest the reference characteristic may be identified. In a further example, adjacent associated value pairs on either side of the reference characteristic may be identified, and an interpolated associated value pair coincident with the reference characteristic may be determined. In a further example, multiple associated value pairs may be identified (e.g., each corresponding to one period's worth of the first segment) based on the comparison (e.g., intersections with the reference characteristic), and a histogram may be generated based on the multiple pairs.

In some embodiments, step 812 may include selecting, or otherwise generating, the reference characteristic. In some embodiments, the reference characteristic may depend on a physiological rate. For example, different reference characteristics may be used for small heart rates relative to large heart rates. In some embodiments, the system may select a reference characteristic such as a curve that is perpendicular to the attractor. In some embodiments, the system may select a reference characteristic such as a curve that is located at an optimal location relative to the attractor. For example, the system may generate a curve located where the cycle to cycle spread of the attractor is at a minimum or an expected minimum.

In some embodiments, a reference characteristic may include a pattern, a template, a set of logical rules, any other suitable reference against which the associated value pairs may be compared, or any combination thereof. For example, the reference characteristic may include a template corresponding to a loop in an attractor, and the associated value pairs may be compared with the template to identify associated value pairs corresponding to a loop. In a further example, the processing equipment may identify looped zero crossings of the attractor, which may correspond to dicrotic notches in the PPG signal, to characterize the number and/or size of notches in the PPG signal.

Step 814 may include the processing equipment determining a signal quality metric based on the comparison of step 812. The signal quality metric may be indicative of variability, or the lack of variability thereof, in the PPG signal from cycle to cycle. In some embodiments, at step 814 the processing equipment may identify intersections between the associated value pairs and the reference characteristic, and determine a mean location (e.g., center location of a histogram peak), standard deviation (e.g., standardized deviation of the histogram peak from the center), median deviation, entropy, a number of intersections, any other suitable metric derived from the comparison of step 812, or any combination thereof, as a measure of signal quality. For example, the processing equipment may determine the mean location of a histogram peak and compare it with predetermined thresholds to determine a signal quality metric. In a further example, the processing equipment may determine the standard deviation of a generated histogram and compare it with a threshold. If the standard deviation exceeds the threshold, the processing equipment may categorize the PPG signal as having low quality. In a further example, a reference characteristic may include a line segment, and the processing equipment may determine a number of intersections with the attractor to determine a signal quality metric (e.g., the line segment may be located at an expected intersection and a larger number may indicate stability). In a further example, a generated histogram may be compared with a reference distribution, and a similarity metric may be determined. In a further example, a determined metric itself may be used as a signal quality metric. In a further example, a determined metric may be scaled and used as a signal quality metric. In a further example, a determined metric may be input into a function or look-up table which outputs a signal quality metric.

In some embodiments, the system may output the signal quality metric for display to a user, for example, in the form of a number, bar, icon, or other indicator.

In some embodiments, the system may use the signal quality metric to assist in determining physiological information (e.g., pulse rate, oxygen saturation, CNIBP, fluid volume, respiration rate, respiration effort). For example, physiological data may be disqualified if a signal quality metric is too low (e.g., less than a threshold). In a further example, the filtering or processing applied by the system to the physiological data may change depending on the signal quality metric. In some embodiments, the system may vary filtering of physiological parameters based on the signal quality metric. For example, for physiological data corresponding to low signal quality, the most recently calculated physiological parameter may be weighted less when averaging it with previously calculated values (e.g., using a digital filter).

In an illustrative example of the techniques of flow diagram 800, FIG. 9 shows a plot of illustrative attractor 700 of FIG. 7 and a reference characteristic 900, in accordance with some embodiments of the present disclosure. Reference characteristic 900 is a vertical line segment (e.g., given by equation x=0). Vertical and horizontal lines, or segments thereof, may provide convenient reference characteristic, because the identification of intersections (e.g., intercepts) is relatively easy. For example, referencing FIG. 9, the intersections of the attractor 700 (i.e., associated value pairs) are the “y-intercepts” and may be identified by finding zeros or near zeros in the first value of each associated value pair. However it will be understood that any suitable curve, linear or otherwise, of any suitable orientation, may be used as a reference characteristic. In some embodiments, more than one reference characteristic may be used. For example, a second vertical line may be added to FIG. 9 (not shown), and a further subset of associated value pairs corresponding to intersections with the second line may be identified. Any suitable number of reference characteristics may be used in accordance with the present disclosure. FIG. 10 is a histogram 1000 of intersection locations of attractor 700 and reference curve 900 of FIG. 9, in accordance with some embodiments of the present disclosure. The abscissa of FIG. 10 is in units along curve 900, while the ordinate of FIG. 10 is the number of intersections. PPG signals having relatively low variation from pulse wave to pulse wave are expected to exhibit a tighter histogram (e.g., a higher peak and smaller spread), while PPG signals having greater variability are expected to exhibit a wider, more spread out histogram. In some embodiments, based on a histogram such as histogram 1000, the processing equipment may determine a mean location, standard deviation, median deviation, entropy, a number of intersections, any other suitable metric, or any combination of thereof, as a measure of signal quality. In some embodiments, the processing equipment may compare a generated histogram such as histogram 1000 with a reference distribution, and a similarity metric may be determined and used as a signal quality metric.

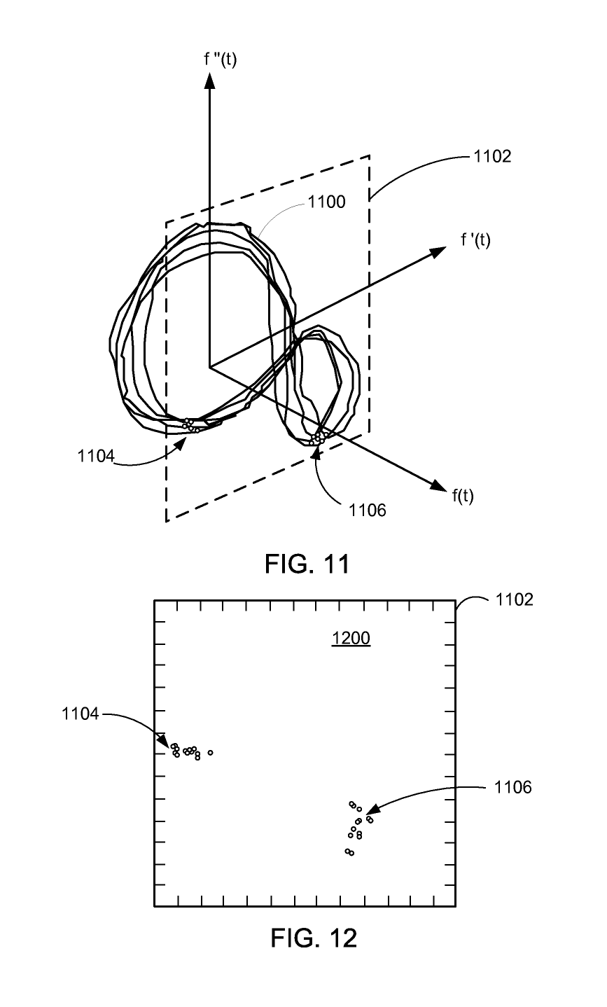

In some embodiments, the processing equipment may generate an attractor in more than two dimensions. For example, the processing equipment may generate associated value groups of more than two (e.g., associated value triples). FIG. 11 shows a plot of an illustrative attractor 1100 in three-dimensions generated using associated value triples, in accordance with some embodiments of the present disclosure. Attractor 1100 includes associated value triples, in which the first value is f(t), the second value is f′(t), and the third value is f″(t), where:

$$\begin{matrix}{f}^{′} ( t ) = \frac{d f}{d t} & ( 16 ) \\ {f}^{″} ( t ) = \frac{{d}^{2} f}{d {t}^{2}} & ( 17 )\end{matrix}$$

where f′(t) is the value of the first derivative (i.e., first order) of the PPG signal f(t) at time t, and f′(t) is the value of the second derivative (i.e., second order) of the PPG signal at time t. The first and second derivatives are illustrative, and any suitable difference signal, of any suitable order may be used. For example, the processing equipment may approximate Eqs. 16-17 using respective difference equations. In a further example, the processing equipment may approximate the PPG signal with a continuous function and use the continuous forms of Eqs. 16-17 to determine the first and second difference signals. In some embodiments, if an attractor is generated in more than two dimensions, the reference characteristic may accordingly be defined in higher dimensions. For example, where a line may be used as a reference characteristic in two dimensions, a plane may be used as a reference characteristic in three dimensions. Surface 1102 is a planar reference characteristic that intersects attractor 1100 at regions indicated by arrows 1104 and 1106. FIG. 12 shows intersections of attractor 1100 and the reference characteristic 1102, viewed normal to reference characteristic 1102 (i.e., in the −f(t) direction), in accordance with some embodiments of the present disclosure. In the illustrated example, two-dimensional space 1200 is coincident with reference characteristic 1102. The two groupings of intersections, indicated by open circles in FIG. 12, between attractor 1100 and reference characteristic 1102 are indicated by arrows 1104 and 1106. Any of the illustrative techniques of flow diagram 800 may be applied in dimensions greater than two. For example, the processing equipment may determine a mean location, standard deviation, median variance, entropy, distribution, any other suitable metric or result from which a metric may be derived, or any combination thereof, for each grouping of intersections indicated by arrows 1104 and 1106. For example, for each group of intersections, the processing equipment may determine the root mean square (RMS) distance of the intersections from the mean location and compare the RMS distance to a threshold. In a further example, for each group of intersections, the processing equipment may determine the mean location of the intersections from the mean location and compare the mean location to a reference location. In a further example, for each group of intersections, the processing equipment may determine the entropy of the intersections and compare the entropy to a reference location. In a further example, for each group of intersections, the processing equipment may determine a distribution of intersections about a mean location and compare the distribution to a reference distribution.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining signal quality of a physiological signal, the method comprising:
receiving a photoplethysmograph signal;
generating, using processing equipment, a difference signal based on the photoplethysmograph signal;
specifying, using the processing equipment, a segment of the photoplethysmograph signal comprising a first plurality of values;
specifying, using the processing equipment, a segment of the difference signal comprising a second plurality of values;
associating, using the processing equipment, each value of the first plurality of values with a corresponding value of the second plurality of values to generate a plurality of associated value pairs;
comparing, using the processing equipment, the plurality of associated value pairs to a reference characteristic; and
determining, using the processing equipment, a signal quality metric based on the comparison.

2. The method of claim 1, wherein the reference characteristic comprises a set of reference value pairs obeying a linear relationship, and wherein comparing the plurality of associated value pairs to the reference characteristic comprises determining a set of resultant values that are nearest to the reference value pairs.

3. The method of claim 2, wherein determining the signal quality metric comprises determining a variability metric among the resultant values.

4. The method of claim 3, wherein the determining the variability metric comprises generating a histogram.

5. The method of claim 3, wherein the variability metric is selected from the group comprising an average magnitude, a standard deviation, a median deviation, and an entropy value.

6. The method of claim 1, wherein the difference signal comprises a first derivative of the photoplethysmograph signal.

7. The method of claim 1, further comprising normalizing at least one of the first plurality of values and the second plurality of values prior to comparing the plurality associated value pairs to the reference characteristic.

8. The method of claim 1, wherein the reference characteristic comprises a reference pattern of values.

9. The method of claim 1, further comprising:
generating a second difference signal based on the photoplethysmograph signal;
specifying a segment of the second difference signal comprising a third plurality of values; and
associating each of the plurality of associated value pairs with a corresponding value of the third plurality of values to generate a plurality of associated value triples;
wherein comparing the plurality of associated value pairs to the reference characteristic comprises comparing the plurality of associated value triples to the reference characteristic.

10. The method of claim 9, wherein the reference characteristic comprises a set of reference value triples obeying a linear relationship, and wherein determining the set of the resultant values comprises determining a set of resultant value triples that are nearest to the reference value triples.

11. A system for determining signal quality of a physiological signal, the system comprising:
processing equipment configured to:
receive a photoplethysmograph signal;
generate a difference signal based on the photoplethysmograph signal;
specify a segment of the photoplethysmograph signal comprising a first plurality of values;
specify a segment of the difference signal comprising a second plurality of values;
associate each value of the first plurality of values with a corresponding value of the second plurality of values to generate a plurality of associated value pairs;
compare the plurality of associated value pairs to a reference characteristic; and
determine a signal quality metric based on the comparison.

12. The system of claim 11, wherein the reference characteristic comprises a set of reference value pairs obeying a linear relationship, and wherein the processing equipment is further configured to determine a set of resultant values that are nearest to the reference value pairs.

13. The system of claim 12, wherein the processing equipment is further configured to determine a variability metric among the resultant values.

14. The system of claim 13, wherein the processing equipment is further configured to generate a histogram.

15. The system of claim 13, wherein the variability metric is selected from the group comprising an average magnitude, a standard deviation, a median deviation, and an entropy value.

16. The method of claim 11, wherein the difference signal comprises a first derivative of the photoplethysmograph signal.

17. The system of claim 11, wherein the processing equipment is further configured to normalize at least one of the first plurality of values and the second plurality of values prior to comparing the plurality associated value pairs to the reference characteristic.

18. The system of claim 11, wherein the reference characteristic comprises a reference pattern of values.

19. The system of claim 11, wherein the processing equipment is further configured to:
generate a second difference signal based on the photoplethysmograph signal;
specify a segment of the second difference signal comprising a third plurality of values; and
associate each of the plurality of associated value pairs with a corresponding value of the third plurality of values to generate a plurality of associated value triples;
wherein the processing equipment is further configured to compare the plurality of associated value triples to the reference characteristic.

20. The system of claim 19, wherein the reference characteristic comprises a set of reference value triples obeying a linear relationship, and wherein the processing equipment is further configured to determine a set of resultant value triples that are nearest to the reference value triples.
